(19) United States
(12) Patent Application Publication
(10) Pub. No.: US 2011/0076235 A1
(43) Pub. Date: Mar. 31, 2011

(54) GENES, METHODS, AND COMPOSITIONS RELATED TO NEUROGENESIS AND ITS MODULATION

(76) Inventors: Timothy P. Tully, Solana Beach, CA (US); Hollis T. Cline, Solana Beach, CA (US)

(21) Appl. No.: 12/893,236

(22) Filed: Sep. 29, 2010

Related U.S. Application Data
(60) Provisional application No. 61/246,967, filed on Sep. 29, 2009.

(51) Int. Cl. A61K 49/00; C12Q 1/68; C12Q 1/02; C12Q 1/06; C07H 21/00; C07H 21/02; A61K 31/7088; A61K 31/713; A61P 25/00

(52) U.S. Cl. 424/9.2; 435/6; 435/29; 435/39; 536/24.5; 514/44 A

(57) ABSTRACT

The present disclosure provides methods for investigating neurogenesis, neural cell proliferation and differentiation. Specifically, the present disclosure relates to methods for identifying pharmaceutical agents capable of modulating neurogenesis and neural cell proliferation, methods of screening for genes that modulate neurogenesis and proliferation of neural progenitor cells, and methods of identifying pharmaceutical agents as candidate modulators of neurogenesis and neural proliferation or differentiation. The present disclosure also relates to methods for identifying pharmaceutical agents to characterize and modulate neurogenesis, pharmaceutical agents identified by such methods, methods for treating patients with such pharmaceutical agents, and compositions containing such pharmaceutical agents. Accordingly, the present methods enable elucidation of the mechanisms that control neurogenesis, brain development and function in healthy animals and in disorders of the nervous system. Furthermore, the present methods facilitate the development of compositions to prevent, improve or stabilize impaired neurogenesis in various nervous system disorders, including cognitive disorders.

### RELATED APPLICATIONS

The present application claims priority under 35 U.S.C. §119 to U.S. Provisional Pat. App. No. 61/246,967, filed on Sep. 29, 2009, which is expressly incorporated herein by reference in its entirety.

### SEQUENCE LISTING

The instant application contains a Sequence Listing which has been submitted in ASCII format via EFS-Web and is hereby incorporated by reference in its entirety. Said ASCII copy, created on Sep. 28, 2010, is named 21RE6550.txt and is 2,011,425 bytes in size.

### FIELD OF THE INVENTION

The present invention relates to genes, methods, and compositions involved in neurogenesis, particularly activity-dependent modulation of neurogenesis in the central nervous system. More particularly, the present invention relates to methods for identifying and manipulating genes involved in neurogenesis and for screening and evaluating pharmaceutical agents that modulate neurogenesis.

### BACKGROUND OF THE INVENTION

Neurogenesis is a complex process that underlies the development and maturation of the nervous system. This process is dependent on proper spatiotemporal regulation of cell proliferation, survival, differentiation and migration. Newly produced nerve cells are able to differentiate into functional cells of the central nervous system and integrate into neural circuits in the brain. Moreover, in the brains of many animals, new nerve cells are continuously generated throughout the life span of the organism. For example, neurogenesis is now known to persist throughout adulthood in two regions of the mammalian brain: the subventricular zone (SVZ) of the lateral ventricles and the dentate gyrus of the hippocampus. In these regions, multipotent neural progenitor cells (NPCs) continue to divide and give rise to new functional neurons and glial cells (Jacobs, Mol. Psychiatry 2000, 5(3): 262-9). Control of neurogenesis therefore underlies the regional specialization of the CNS and the establishment of specific cell types that make up functional circuits.

Neurogenesis plays a fundamental role in CNS physiology. The pool of neural progenitor cells (NPCs) can be expanded by symmetric divisions that give rise to additional NPCs or depleted by terminal differentiation of progeny into neurons or glia (Butt et al., Neuron 2005, 48: 591-604; Gotz et al., Nat Rev Mol Cell Biol 2005, 6: 777-788; Huttner et al., Curr Opin Cell Biol 2005, 17: 648-657; Noctor et al., Arch Neurol 2007, 64: 639-642; Kriegstein et al., Ann Rev Neurosci 2009, 32: 149-184). Maintaining NPCs in an actively proliferative state can therefore enlarge the progenitor pool and can enlarge the cerebral cortex of mice (Chenn et al., Science 2002, 297: 365-369; Lehmann et al., Eur J Neurosci 2005, 21: 3205-3216). Conversely, premature transition of NPCs from a proliferative to differentiated state can deplete the progenitor pool; in the short-term, this can increase the generation of differentiated neurons and glia, but in the long-term, depletion of the progenitor pool limits future generation of neural progeny.). Cortical size can also be altered by increasing or decreasing the survival of NPCs. (Depaepe et al., Nature 2005, 435: 1244-1250; Putz et al., Nat. Neurosci. 2005, 8: 322-331).

Disruptions in neurogenesis can play a fundamental role in diseases and disorders of the CNS. Neuronal cell number is largely determined by proliferation of NPCs and the survival and differentiation of their progeny. While these steps can be regulated independently, they also must be properly coordinated to establish properly functioning circuits within the nervous system. Indeed, errors in the generation of neurons and their assembly into circuits can lead to numerous neurological disorders including heterotopias, mental retardation, autism spectrum disorders, epilepsy, focal cortical dysplasia. Alterations in the production of neuronal cell types can lead to a disproportionate ratio of excitatory and inhibitory circuits in the brain, an imbalance that may underlie autism spectrum disorders, depression and schizophrenia.

These observations underscore the crucial role of mechanisms modulating NPC proliferation and the fate of their progeny. Mechanisms that regulate cell proliferation can be classified as cell-autonomous or non-cell-autonomous. With respect to cell-autonomous mechanisms, tremendous progress has recently been made in identifying factors required for pushing cells into a ‘pluripotent/multipotent’ state and for maintaining this state. (Welstead et al., Curr. Opin. Genet. Dev. 2008, 18: 123-129).

Less well-understood, however, are non-cell autonomous mechanisms, which depend on neural activity in vivo. Elucidating the mechanisms that control neurogenesis in intact animals is therefore crucial to a proper understanding of brain development and function in healthy animals and in disorders of the nervous system. It is also crucial to the development of methods and compositions to prevent, improve and/or stabilize neurogenesis (e.g., modulate), and specifically impaired neurogenesis, in the nervous system and specifically in nervous system disorders, including cognitive disorders.

The present invention meets these and other needs in the art by providing targets, methods, and compositions that relate to the modulation of neurogenesis in an activity dependent manner.

### SUMMARY OF THE INVENTION

In a first aspect, the present disclosure relates to a method comprising contacting neural progenitor cells in an intact brain region of a first animal with a pharmaceutical agent, exposing the first animal and a second control animal to an external stimulus capable of eliciting activity in the intact brain region, and measuring proliferation rates of the neural progenitor cells in the first animal and of neural progenitor cells in the second animal, in any order, wherein a difference in proliferation rate between the neural progenitor subject cells and the neural progenitor control cells identifies the pharmaceutical agent as one capable of modulating neural proliferation.

In certain embodiments, the first and second animals may be vertebrates, including amphibians and mammals. More particularly, the first and second animals may be Xenopus laevis, and more specifically may be tadpoles of Xenopus laevis.

In certain embodiments, the intact brain region may be involved in processing olfactory inputs, visual inputs, or mechanosensory inputs, or may be involved in mediating behavioral outputs. In specific embodiments, the first and second animals may be Xenopus laevis and the intact brain region may be the optic tectum. The intact brain region can also comprise circuits of the telencephalon, midbrain, hindbrain/spinal cord, retina, or olfactory pit.

In certain embodiments, measuring the proliferation rates of the neural progenitor cells in the experimental and control animals comprises counting the number and type of cells in the optic tectum of the first and second animals.

In some embodiments, contacting the neural progenitor cells with pharmaceutical agent may comprise electroporating said pharmaceutical agent into said neural progenitor cells.

In another aspect, the present disclosure relates to a method comprising contacting neural progenitor subject cells with a pharmaceutical agent in an amount effective to modulate expression of one or more genes in said neural progenitor subject cells, measuring proliferation rates of the neural progenitor subject cells and of neural progenitor control cells that have not been contacted with the pharmaceutical agent, and comparing the proliferation rates of the neural progenitor subject cells and the neural progenitor control cells, in any order, wherein a difference in proliferation rate between the neural progenitor subject cells and the neural progenitor control cells identifies the one or more genes as modulators of proliferation of neural progenitor cells.

In certain embodiments, the neural progenitor subject cells may be in a first animal and the neural progenitor control cells may be in a second animal. In some embodiments, the neural progenitor subject and control cells may be in the optic tectum of each of the first and second animals respectively. In one embodiment, the first and second animals may be Xenopus laevis.

In some embodiments, the method may further comprise introducing a reporter construct into the neural progenitor subject cells and the neural progenitor control cells. In certain embodiments, the reporter construct may comprise a gene encoding a fluorescent protein. In one embodiment, expression of the fluorescent protein may be restricted spatially, in particular to a specific cell type, such as neural progenitor cells. In another embodiment, expression of the fluorescent protein may also be restricted temporally, for example, restricted to progeny cells produced in a brain region after a particular point in time, In other embodiments, introducing the a reporter construct into the neural progenitor subject cells may comprise transfecting the cells with a plasmid encoding the reporter construct.

In some embodiments, measuring the proliferation rates of the neural progenitor cells may comprise counting the number and type of cells before and after at least one predetermined time period.

In another embodiment, the method may further comprise exposing the first and second animals to a visual stimulus.

In some embodiments, the pharmaceutical agent may comprise a chemical compound or an antisense oligonucleotide. In certain embodiments, the antisense oliogonucleotide may comprise an siRNA, an shRNA and/or a morpholino.

In another embodiment of the present method, the one or more genes in the neural progenitor subject cells may be selected from SEQ. ID. NOs. 1-651, or functional truncations, modifications and/or substitutions thereof.

In another aspect, the present disclosure relates to a method comprising contacting neural progenitor subject cells with a pharmaceutical agent, measuring proliferation rates of the neural progenitor subject cells and of neural progenitor control cells that have not been contacted with the pharmaceutical agent, comparing the proliferation rates of the neural progenitor subject cells and the neural progenitor control cells, in any order, wherein a difference in proliferation rate between the neural progenitor subject cells and the neural progenitor control cells identifies the pharmaceutical agent as one capable of modulating proliferation.

In some embodiments, the method may comprise introducing a reporter construct into the neural progenitor subject cells and the neural progenitor control cells. In certain embodiments, the reporter construct may comprise a gene encoding a fluorescent protein. In some embodiments, the fluorescent protein may be specifically expressed in neural progenitor cells.

In some embodiments, introducing the reporter construct into the neural progenitor cells comprises transfecting neural progenitor cells with a plasmid encoding the reporter construct.

In some embodiments, contacting the neural progenitor subject cells with a pharmaceutical agent comprises electroporating the pharmaceutical agent into the neural progenitor subject cells.

In certain embodiments, the method may further comprise exposing the first and second animals to a visual stimulus.

In a fourth aspect, the present disclosure relates to a method comprising administering a pharmaceutical agent to subject cells expressing a target gene selected from the group consisting of SEQ ID NOs. 1-651, or functional truncations, modifications and/or substitutions thereof, comparing expression of the target gene in the subject cells administered the pharmaceutical agent compared with expression of the target gene in subject cells not administered the pharmaceutical agent, in any order, wherein a difference in expression of the target gene in subject cells administered the pharmaceutical agent compared with subject cells not administered the pharmaceutical agent identifies the pharmaceutical agent as a candidate modulator of neural proliferation or differentiation.

In another aspect, the present disclosure relates to pharmaceutical agents identified by the methods described herein.

In still another aspect, the present disclosure relates to pharmaceutical compositions comprising a pharmaceutical agent identified by the methods described herein.

In still another aspect, the present disclosure relates to methods for treating a patient comprising administering compounds identified be by the methods described herein.

The present invention comprises methods for investigating the phenomenon of neural cell proliferation and differentiation, pharmaceutical agents identified by such methods, compositions containing the same and methods of treatment comprising administering such pharmaceutical agents or compositions. Accordingly, the present disclosure provides methods of identifying genes implicated in the regulation of neurogenesis, methods for identifying pharmaceutical agents to characterize and modulate neurogenesis, and modulators and/or treatments for the nervous system and specifically various nervous system disorders and/or injuries including methods and compositions to prevent, improve and/or stabilize neurogenesis (i.e., modulate), and specifically impaired neurogenesis, in the nervous system and specifically in nervous system disorders, including cognitive disorders. The present invention also comprises pharmaceutical agents selected by the methods of the present invention, as well as pharmaceutical compositions comprising such selected pharmaceutical agents, as well as methods of administering such pharmaceutical agents and compositions to patients, wherein patients include human patients and wherein said administering is for the purpose of modulating neurogenesis and specifically to prevent, improve and/or stabilize neurogenesis and specifically impaired neurogenesis, in the nervous system and specifically in nervous system disorders, including cognitive disorders, in patients and specifically in humans.

### BRIEF DESCRIPTION OF THE FIGURES

FIG. 1 depicts the transparent brain of a Xenopus laevis tadpole (A); the optic tectum region of the brain (B); and proliferation and differentiation of cells within the tectal lobe (C).

FIG. 2 is a diagram showing the different lineages of neural progenitor cells (NPCs).

FIG. 3 is a diagram of a proliferation reporter that facilitates spatial and temporal resolution of labeled cells imaged in the intact brain.

FIG. 4 is an image showing the proliferation of NPCs over a 24-hour period in the optic tectum of Xenopus laevis.

FIG. 5 is a graph showing decreased proliferation rate of tectal cells following exposure to cell division blockers.

FIG. 6 is graph showing tectal proliferation rates in the presence or absence of cell division blockers on day 1 without visual stimulation and day 2 with visual stimulation (A); and the percent of tectal neurons in the presence or absence of cell division blockers on day 3 (B).

FIG. 7 is a graph showing tectal proliferation rates in animals expressing a morpholino against Deiodinase iodothyronine type III (dio3-MO) with and without visual stimulation, compared to control animals.

FIG. 8 is a graph showing the percent of tectal neurons and glial cells in animals expressing a morpholino against Glutathione S-transferase Pi 1 (GSTpi-MO) over three days, compared to control animals.

FIG. 9 is a graph showing tectal proliferation rates (A) and the percent of tectal neurons and glial cells (B) in animals expressing a morpholino against one of 11 genes of interest (GOIs) over three days, compared to control animals. GOIs: Heat shock protein 70 (HSPA5); Ephrin receptor type B-1 (EphRB1); Deiodinase iodothyronine type III (Dio3); ETS domain-containing protein Elk-4 (ELK4); Wingless-type MMTV (murine mammary tumor virus) integration site family, member 7b (Wnt7b); Fragile X mental retardation, autosomal homolog (FXR1); Fragile X mental retardation protein 1 (FMR1A); Matrix metallopeptidase 9 (MMP9), cAMP-dependent protein kinase catalytic subunit alpha (PRKACA), Neuritin 1-A (cpg15); Glutathione S-transferase Pi 1 (GSTPi).

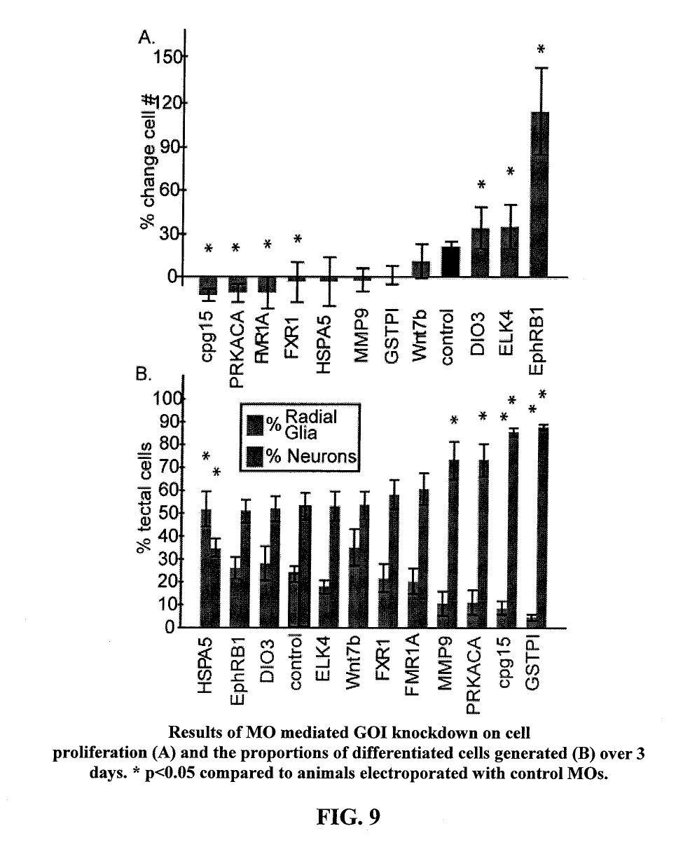

### DETAILED DESCRIPTION OF THE INVENTION

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described herein. All publications, patent applications, patents, and other references mentioned herein are incorporated by reference in their entirety. In addition, the materials, methods, and examples are illustrative only and are not intended to be limiting.

Reference is made to standard laboratory manuals that contain definitions and methods and means for carrying out basic techniques encompassed by the present invention. See, for example, Sambrook et al., Molecular Cloning: A Laboratory Manual, Third Edition, Cold Spring Harbor Laboratory Press, New York (2001); Current Protocols in Molecular Biology, Ausubel et al. (eds.), John Wiley & Sons, Inc., Hoboken, N.J. (2010), Current Protocols in Cell Biology, Bonifacino et al. (eds.), John Wiley & Sons, Inc., Hoboken, N.J. (2010); Current Protocols in Immunology, Coligan et al. (eds,), John Wiley & Sons, Inc., Hoboken, N.J. (2010); Current Protocols in Neuroscience, Gerfen et al. (eds.), John Wiley & Sons, Inc., Hoboken, N.J. (2010); Current Protocols in Nucleic Acid Chemistry, Egli et al. (eds.), John Wiley and Sons, Inc., Hoboken, N.J. (2010); Current Protocols in Pharmacology, Enna et al. (eds.), John Wiley and Sons, Inc., Hoboken, N.J. (2010); and the various references cited therein.

### Definitions

As used in the present disclosure, the term “about” or “approximately” means within an acceptable range for a particular value as determined by one skilled in the art, and may depend in part on how the value is measured or determined, e.g., the limitations of the measurement system or technique.

The terms “a,” “an” and “the” are to be understood as meaning both singular and plural, unless explicitly stated otherwise. Thus, “a,” “an,” and “the” (and grammatical variations thereof where appropriate) generally refer to one or more.

As used herein, “neurogenesis” includes the proliferation, survival, differentiation, and migration of a neural cell in vivo, in vitro, or ex vivo. The cells may be located in or obtained or originated from the central nervous system or elsewhere in an animal or human being (e.g., the peripheral nervous system). Neurogenesis is intended to include neurogenesis as it occurs during normal development, as well as neural regeneration that occurs following disease, damage or therapeutic intervention. Embodiments of the disclosed invention include the detection or measurement of either proliferation or differentiation as non-limiting indicators of neurogenesis.

An “external stimulus” is broadly defined to encompass any type of simple or complex extracellular stimulus that can induce neural activity. Thus an external stimulus includes inputs to the visual system of an animal. It also includes input to other brain regions, such as those involved in processing olfactory, mechanosensory, or visual inputs, and in mediating behavioral outputs.

The term “modulate” as used herein includes altering the expression of a gene, or level of RNA molecules or equivalent RNA molecules, including non-coding RNAs and those encoding one or more proteins or protein subunits. “Modulate” also includes altering activity of one or more gene products (including non-coding RNAs), proteins, or protein subunits, such that expression, level, or activity in the presence of a modulator differs from that observed in the absence of a modulator. For example, the term “modulate” can mean “up-regulate” or “down-regulate,” although use of the word “modulate” is not limited to these definitions. Modulation can be an increase or a decrease in expression or activity, a change in binding characteristics of a gene product, or any other change in the biological, functional, or immunological properties of biologically active molecules.

“Modulation of neurogenesis” or “modulating neurogenesis” includes changes in cell proliferation, survival, differentiation, or migration. Such a change can occur in a cell or population of cells, including those within an intact brain region. Non-limiting examples include increased (or decreased) levels of an inducer (or inhibitor) of neurogenesis, such as changes in the level of a gene product directly involved in NPC proliferation. Such changes may also include a difference in cell differentiation or cell migration within a neural circuit. In certain embodiments, modulating neurogenesis refers to effects on cell proliferation and on cell fate (e.g., neuronal versus glial).

The terms “modulator,” “compound” and “pharmaceutical agent” may be used interchangeably herein, and include pharmacologically active substances in isolated form, or mixtures thereof. For example, a pharmaceutical agent, compound or modulator may be an isolated and structurally-defined product, an isolated product of unknown structure, a mixture of several known and characterized products, or an undefined composition comprising one or more products. Examples of such undefined compositions include for instance tissue samples, biological fluids, cell supernatants, vegetal preparations, etc. The pharmaceutical agent, compound or modulator may be any organic or inorganic product, including a polypeptide (or a protein or peptide), a nucleic acid, a lipid, a polysaccharide, a chemical entity, or mixture or derivatives thereof. The pharmaceutical agent, compound or modulator may be of natural or synthetic origin, and the compound(s) or modulators may include libraries of compounds.

A “modulator,” “compound,” or “pharmaceutical agent” can increase (or decrease) the amount, degree, or nature of a neurogenic response in vivo, in vitro, or ex vivo, relative to the amount, degree, or nature neurogenesis in the absence of the agent or reagent. In certain embodiments, treatment with such a “neurogenic” agent may increase (or decrease) the amount, degree, or nature of a neurogenic response by at least about 1%, 2%,3%, 4%, 5%, 10%, 20%, 40%, 50%, 75%, 100%, 200% (2 fold), 300% (3 fold), 400% (4 fold), 500% (5 fold), or still more or less, compared to the amount, degree, or nature or a neurogenic response in the absence of the agent, under the conditions of the method used to detect or determine neurogenesis.

The terms “inhibit,” “down-regulate,” or “reduce” include decreasing expression of a gene, or level of RNA molecules or equivalent RNA molecules encoding one or more proteins or protein subunits, or activity of one or more gene products, proteins or protein subunits below that observed in the absence of one or more modulators (e.g., siRNA, shRNA, antisense morpholino, etc.) as defined in the claimed methods. The terms “enhance” or “up-regulate” includes increasing expression of a gene, or level of RNA molecules or equivalent RNA molecules encoding one or more proteins or protein subunits, or activity of one or more gene products, proteins or protein subunits below that observed in the absence of one or more modulators as defined in the claimed methods.

The term “target gene” or “gene or interest” includes not just protein-coding genes but non-coding genes. Such non-coding genes include those encoding ribosomal RNAs (rRNAs), transfer RNAs (tRNAs), and small nuclear RNAs (snRNAs), as well as microRNAs, snoRNAs, siRNAs , piRNAs, and ncRNAs. It can also include a polynucleotide region that regulates replication, transcription, translation, or other processes important to expression of the gene product, or a polynucleotide comprising both a region that encodes a gene product and a region operably linked thereto that regulates expression. The targeted gene may be chromosomal (genomic) or extrachromosomal. It may be endogenous to the cell, or it may be a foreign gene (a transgene). The foreign gene may be integrated into the host genome, or it may be present on an extrachromosomal genetic construct such as a plasmid or a cosmid. The targeted gene may also be derived from a pathogen, such as a virus, bacterium, fungus or protozoan, which is capable of infecting an organism or cell. Target genes may be viral and pro-viral genes. In a specific embodiment, a target gene is one involved in or associated with the progression of cellular activities important to neurogenesis.

The term “target nucleic acid” includes any nucleic acid sequence whose expression or activity is to be modulated. The target nucleic acid may be DNA or RNA. Furthermore, the target gene or gene may comprise fragments of larger nucleic acid sequences that are generally biologically active.

Reference will now be made to the several embodiments of the present disclosure, examples of which are illustrated by and described in conjunction with the accompanying drawings and examples. While certain embodiments are described herein, it will be understood that the described embodiments are not intended to limit the scope of the invention as defined by the appended claims. On the contrary, the present disclosure is intended to cover alternatives, modifications and equivalents that may be included within the spirit and scope of the invention as defined by the appended claims. Furthermore, in the present disclosure certain details are provided to convey a thorough understanding of the invention defined by the appended claims. However, it will be apparent to those skilled in the art that certain embodiments may be practiced without these details. In certain instances, well-known methods, procedures, or other specific details have not been described to avoid unnecessarily obscuring aspects of the invention defined by the appended claims.

### Methods of Identifying and Characterizing Genes Involved in Neurogenesis

Xenopus laevis as a Model System

Xenopus laevis has proven advantageous for in vivo studies of neurogenesis and brain development. Several factors underlie these advantages:

Compact Period of Neurogenesis

Frog tadpoles have a relatively prolonged and accessible period of cell proliferation and differentiation that extend through the larval period of CNS development. Over the course of the pre-metamorphic stages of development of X. laevis, new neurons are generated via cell proliferation. The newly formed neurons then integrate into the functional circuitry of the developing tadpole brain. The neurogenic sequence from birth to differentiation of individual neurons can be captured in a 2-4 day period in X. laevis, as opposed to over a month in mammalian systems.

This compact time frame facilitates detailed investigations into the different steps comprising neurogenesis. This advantage, together with the evidence that mechanisms of neurogenesis are evolutionarily conserved, underscores the value of Xenopus not only in revealing fundamental mechanisms of neurodevelopment of relevance to mammalian systems but also in providing an experimental model system for studying human neurodevelopmental diseases.

Accessible Brain Circuitry

Studies on neuronal development and circuit formation in frogs have revealed several fundamental mechanisms of brain development which have subsequently been shown to function in mammals, including humans. The CNS is highly regionalized along the anterior-posterior axis of X. laevis and different CNS regions are composed of specific neural circuits, with unique functions, including sensory input processing, information integration, memory, decision-making, and motor control.

Anterior-posterior patterning of neural progenitors in the ventricular layer of the CNS is fundamental to the subsequent regional specialization of brain function, and is thought to be established by evolutionarily conserved expression patterns of transcription factor families, such as Otx, Pax and Hox in neural progenitors, followed by activation of transcriptional cascades that define regional subsets of progenitors and their neuronal progeny (O'Leary et al., Curr. Opin. Neurobiol. 2008, 18: 90-100; Lichtneckert et al., Adv. Exp. Med. Biol. 2008, 628: 32-41).

Indeed, Hox family transcription factors not only regulate progenitors and cell fate along the anterior-posterior axis from telecephalon to spinal cord, but also specify contacts between neurons in developing circuits by virtue of controlling downstream transcription factor cascades as neurons differentiate. (Dasen et al., Curr. Top. Dev. Biol. 2009, 88: 169-200; Dalla Torre di Sanguinetto et al., Curr. Opin. Neurobiol. 2008, 18: 36-43.)

The embodiments of the present invention encompass methods for analyzing the transcriptome in different brain circuits and cell types. These include the telencephalon, the midbrain and the hindbrain/spinal cord, as well as the retina and olfactory pit. Each of these regions is characterized by distinct sets of NPC progeny. For example, the telencephalon receives and processes olfactory inputs and includes regions includes regions homologous to the hippocampus and basal ganglia, which are involved in memory and movement control (Maier et al., J Chem Neuroanat, 40(1): 21-35; Brox et al., J Comp Neurol 2004, 474(4): 562-77). The midbrain processes mechanosensory and visual inputs (Hiramoto et al., Dev Neurobiol 2009, 69(14): 959-71; Deeg et al., J Neurophysiol 2009, 102(6): 3392-404) and the hindbrain and spinal cord mediate behavioral outputs (Soffe et al., J Physiol 2009, 587(Pt 20): 4829-44; Orger et al., Nat Neurosci 2008, 11(3): 327-33).

The methods of the instant invention not only encompass analysis of distinct brain regions but also specialized cell types within such circuits. Distinct circuits in each brain area are thought to be composed of unique combinations of excitatory and inhibitory neurons with cell-specific transcript composition, which endow the region-specific circuits with particular properties. Accordingly, the methods of the present invention include analysis of the transcriptome of identified neurons, such as GABAergic and glutamatergic neurons in the brain regions described here.

In some embodiments, the methods may be directed to the optic tectum region of X. laevis. The optic tectum is the primary visual center in nonmammalian vertebrates; it is a paired structure that forms a major component of the midbrain (or mesencephalon) and receives inputs from retinal fibers in a topographically ordered manner. See, for example, Dingwell et al., J. Neurobiol. 2000, 44: 246-259.

Direct Imaging

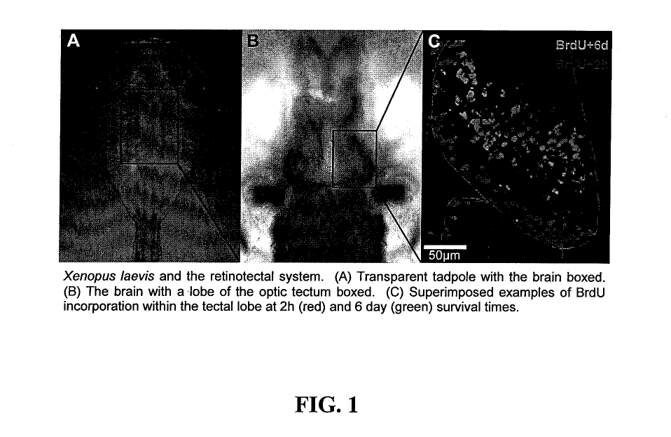

NPC proliferation and differentiation may be directly observed in pre-metamorphic (tadpole stages) Xenopus laevis. FIG. 1A shows the transparent head of a tadpole, with the brain indicated by a box. FIG. 1B is a more detailed view of the region of the brain that includes the optic tectum. FIG. 1C shows the relative location of neural cells (visualized with BrdU) in the optic tectum at 2 hours and at 6 days; newly generated cells differentiate into neurons which migrate away from the ventricular layer. (Wu et al., J. Neuroscience 1999, 19(11): 4472-4483).

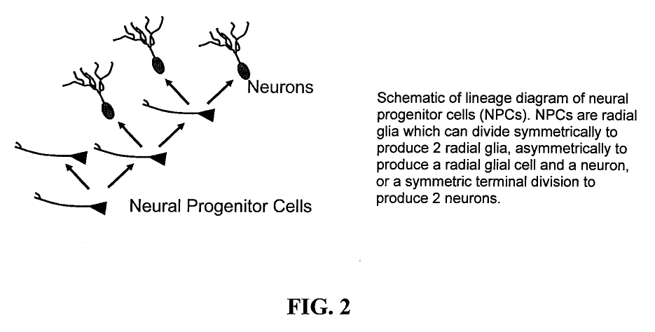

Xenopus tadpoles are therefore amenable to in vivo time-lapse imaging, so that neural progenitor cells (NPCs) and their progeny can be identified and imaged in the intact animal. In this regard, NPCs are undifferentiated radial glia that can divide in distinct modes, as depicted in FIG. 2. In a first mode, a single NPC may divide and form two daughter NPCs (e.g., a “proliferating mode”). In a second mode, a single NPC may divide to form one daughter NPC and one daughter neuron (e.g., a “mixed mode”). In a third mode, a single NPC may divide to form two daughter neurons (a “differentiating mode” or “terminal mode.” See Kriegstein et al., Ann Rev Neurosci 2009, 32: 149-184).

Neurogenic Reporters

Such imaging provides a basis for methods to identify and analyze distinct cell types based on morphology as well as developmental stage. The instant application encompasses the use of multiple cell reporters to facilitates such analyses. For example, such reporters allow tagging and time-lapse monitoring or neural progenitor cells, as well as different populations of differentiated cells, such as GABAergic or glutamatergic neurons

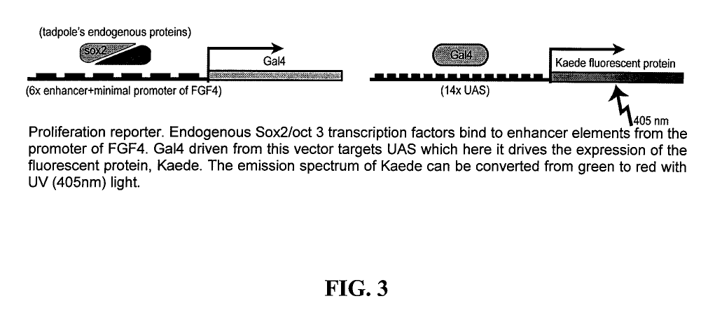

Accordingly, in some embodiments, the present methods may employ a reporter that is specific to dividing NPC cells. For example, the reporter may comprise a binary Gal4-UAS (upstream activation sequence) reporter system (Hartley et al., Proc Nat Acad Sci 2002, 99(3): 1377-1382). FIG. 3 shows an exemplary Gal4-UAS reporter system, which comprises two components: a Gal4 driver and a UAS-reporter. The sequences that control expression of Gal4 will therefore dictate expression of the UAS-reporter.

In the example shown in FIG. 3, the controlling region of Gal4 comprises multiple enhancer elements from the promoter of the fibroblast growth factor 4 (FGF4) gene. Activation of this controlling region requires binding to endogenous sox2/oct3 transcription factors, which are expressed in proliferating NPCs but are not present at significant levels in differentiated, non-proliferating neurons. Hence, this system allows the specific detection of a UAS-reporter in proliferating NPCs.

The reporter may include any marker of interest. In some embodiments, such as that shown in FIG. 3, it may encode a fluorescent protein,(e.g., Kaede fluorescent protein or green fluorescent protein (GFP)), or any other suitable reporter which can be detected or visualized. Accordingly, cells that are actively dividing express the reporter (e.g., Kaede or GFP) protein. In the case of NPCs, daughter cells remaining in the proliferative state, (e.g., are dividing in proliferative mode or in mixed mode (wherein one daughter cell is an NPC) continue to express, for example, the Kaede fluorescent protein. In contrast, division of an NPC in terminal mode will give rise to two differentiated neurons that cease to divide and thus will express little or no Kaede fluorescent protein and will only contain residual Kaede protein inherited during cytokinesis. Accordingly, the above-described Gal4-UAS reporter system can provide a measure of proliferation of NPCs.

The above-described Gal4-UAS reporter system is an exemplary reporter system. However, any suitable reporter system may be employed (e.g., a one-component system or other two-component system). In other embodiments, it may include a gene product that can modulate function in the targeted cell. Indeed, the system allows one to use multiple UAS-reporters in concert with a Gal4 driver.

Moreover, the reporter may have additional properties, such as those allowing temporal resolution of neural cells in vivo. One such reporter is the photoconvertible fluorescent protein Kaede shown in FIG. 3. The Kaede protein exhibits a green fluorescent emission spectrum, but it can be photoconverted to exhibit a red fluorescent emission spectrum upon exposure to either UV light, a 405 nm laser, or any other suitable light source. Accordingly, the photoconvertible property of Kaede enables a temporal control element in observing and characterizing NPC behavior.

For example, NPCs can be transfected with the above-described Gal4-UAS reporter having a Kaede fluorescent effector element. Proliferating cells expressing the reporter will produce daughters with green Kaede protein. After a preferably predetermined period of time (e.g., 1 h, 2 h, 4 h, 8 h, 12, h, 16 h, 20 h, 24 h, 30 h, 36 h, 48 h, 72 h, 96 h, 168 h, or any other value or ranges of values therein), the NPC cells containing Kaede protein may be exposed to a light source (e.g., a 405 nm laser, UV light, etc. . . . ) to photoconvert Kaede protein from green to red. Newly produced progeny would inherit the red Kaede protein during cytokinesis, but those NPCs that remain undifferentiated would produce green Kaede protein (Caron et al., Development 2008, 17: 107-117). Accordingly, newly generated daughter cells may be distinguished from parent cells, and the relative numbers of parent and daughter cells may be determined by measurement of the relative levels of green to red Kaede protein. This measurement may also provide an estimate of the relative proportions of each type of cell division (e.g., proliferation, mixed, terminal).

A reporter system suitable for use in the present methods may be introduced into the cells or tissue of interest by any suitable technique known in the art (e.g., transfection, etc. . . . ). In certain embodiments, the reporter system may be constructed on a plasmid, and may be introduced into the cell via, for example, electroporation (Haas et al., Differentiation 2002, 70: 148-154).

### Differential Screening by Microarray Analysis

In some embodiments, the present methods enable identification of genes involved in neurogenesis, and in particular, NPC proliferation and differentiation. Identification of candidate genes may be performed by, for example, microarray analysis of nucleic acids in radial glia and differentiated neurons, respectively. Such microarray techniques are well known to those in the art. Cells may be selected based on morphology and separated into different populations prior to processing for microarray analysis. For example, cells may be separated based on their characterization as either NPCs or differentiated neurons. Alternatively, cell populations (or animals containing such cell populations of interest) may be exposed to conditions and/or stimuli that either promote or suppress differentiation.

The well-characterized development of the X. laevis tadpole nervous systems provided a basis for identifying candidate genes involved in neurogenesis. For example, genes of interest (e.g., SEQ ID NOs. 1-651), or functional truncations, modifications and/or substitutions thereof, can be identified via comparative microarray analysis of genes expressed in differentiated neurons compared to undifferentiated NPCs in X. laevis (see, e.g., Example 2 below). In one embodiment, cells from the optic tectum of X. laevis are harvested at different days during neurodevelopment, for example at day 1 and day 5. Cells harvested at day 1 will have a larger proportion of undifferentiated NPCs compared to cells harvested at day 5, which will have a larger proportion of differentiated neurons. Genes that showed differential expression and specifically a significant (e.g., p<0.05, 0.04, 0.03, 0.02, 0.01 or less, or any other value therein) differential expression in cells harvested at day 1 relative to those harvested at day 5 represent genes of interest in regulation or modulation of NPC proliferation and differentiation.

In another experimental protocol, cells can be harvested from the brain region of animals that have received or not received an external stimulus of inputs to that region, such as light, an olfactory cue, or mechanosensory stimulation. In one embodiment, the cells are harvested from the optic tectum of animals maintained in the dark and from animals exposed to light. Typically, the cells from animals exposed to dark and light are harvested at the same time (e.g., after 12 hr, 24 hr, 48 hr, 72 hr, etc.) and then microarray analysis of genes contained therein is performed. As discussed herein (see Examples 1 and 2 below), cells harvested from animals maintained in the dark generally have a have a higher proportion of undifferentiated NPCs. Cells exposed to light over the same period generally have a higher relative proportion of differentiated neurons. Accordingly, microarray analysis of these two populations can reveal genes differentially expressed (p<0.01) in cells from animals maintained in the dark (e.g., having a greater relative proportion of NPCs) relative to cells harvested from animals exposed to light, thus identifying those genes as being implicated in NPC proliferation and differentiation.

For example, as described herein, exposure of Xenopus to light has been shown to promote NPC differentiation into neurons in the optic tectum. Accordingly, animals exposed to light may display a higher proportion of differentiated neurons relative to a control animal maintained in the dark. Thus, individual Xenopus animals may be exposed to either dark or light conditions over a period of time (e.g., 12 h, 24 h, 1 day, 2 days, 3 days, 4 days, 7 days, etc. . . . ), and then cells from, for example., the optic tectum, may be collected from the animals and subjected to microarray analysis. Alternatively, animals maintained for longer periods of time will have increasingly developed optic tecta. Accordingly, cells harvested from animals after 24 hours and analyzed via microarray may have a greater proportion of NPCs relative to those harvested from an animal after, for example., 2 days, 3 days, 5 days, 7 days, or more. In either case (light vs. dark or earlier vs. later), the cell populations harvested from the tecta of a first animal population typically have more NPCs and the remaining population from a second animal typically have more differentiated neurons. Accordingly, comparative microarray analysis can reveal those genes preferentially expressed in NPCs. Genes identified by such methods include SEQ ID NO.s 1-651, listed herein. These genes may be preferentially expressed in NPCs, and as such, are implicated in neurogenesis (e.g., NPC proliferation, differentiation and/or survival).

Manipulating Candidate Gene Expression in vivo

The established role of the local environment and non-cell autonomous factors in regulating neurogenesis (Peunova et al., J. Neurosci 2001, 21(22): 8809-8818; Kreigstein et al., 149-84, Cheng et al., Nat Neurosci 2009, 12(4): 399-408; Javaherian et al., Cereb Cortex 2009, 19 Supp 1: i70-77; Suh et al., Ann Rev Cell Dev Biol 2009; 25: 253-75) accentuates the importance of experimental systems, such as Xenopus, in which direct assessment of proliferative activity can be made in the intact animal to identify endogenous cellular and molecular mechanisms regulating neurogenesis.

The use of pharmaceutical agents to modulate gene expression in NPCs in the CNS (e.g., optic tectum) of X. laevis evidences that a gene of interest is implicated in proliferation of NPCs. For example, morpholinos can be designed based on a known gene sequence and effectively silence downstream expressed products of the gene of interest (e.g., RNA, protein). Accordingly, identification of genes that regulate NPC proliferation provides known targets for use in screening pharmaceutical agents that can modulate neurogenesis and NPC proliferation.

### Methods of Screening and Evaluating Modulators of Neurogenesis

### Pharmaceutical Agents

Pharmaceutical agents (drugs), as used herein, include compounds with pharmacological activity and include inorganic compounds, ionic materials, organic compounds, organic ligands, including cofactors, saccharides, recombinant and synthetic peptides, proteins, peptoids, nucleic acid sequences, including genes, nucleic acid products. Pharmaceutical agents can be individually screened. Alternatively, more than one pharamaceutical agent can be tested simultaneously for the ability to modulate neuroactivity or expression of a gene involved in neurogenesis. Where a mixture of pharmaceutical agents is tested, the pharmaceutical agents selected by the methods described can be separated (as appropriate) and identified by suitable methods (e.g., chromatography, sequencing, PCR, etc.

Large combinatorial libraries of pharmaceutical agents (e.g., organic compounds, recombinant or synthetic peptides, peptoids, nucleic acids) produced by combinatorial chemical synthesis or other methods can be tested (see e.g., Zuckerman, R. N. et al., J. Med. Chem., 37: 2678-2685 (1994) and references cited therein; see also, Ohlmeyer, M. H. J. et al., Proc. Nat. Acad. Sci. USA, 90:10922-10926 (1993) and DeWitt, S. H. et al., Proc. Natl. Acad. Sci. USA, 90:6909-6913 (1993), relating to tagged compounds; Rutter, W. J. et al., U.S. Pat. No. 5,010,175; Huebner, V. D. et al., U.S. Pat. No. 5,182,366; and Geysen, H. M., U.S. Pat. No. 4,833,092), the relevant portions of each of which are incorporated herein by reference. Where pharmaceutical agents selected from a combinatorial library carry unique tags, identification of individual pharmaceutical agents by chromatographic methods is possible. Chemical libraries, microbial broths and phage display libraries can also be tested (screened) for the presence of one or more pharmaceutical agent(s) which are capable of modulating neuroactivity or expression of one or more genes involved in neurogenesis in accordance with the methods described herein.

The methods disclosed herein can allow for screening or identification of compounds exhibiting a selected property (e.g., modulating neural progenitor cell proliferation, modulating target gene expression, etc. . . . ). The methods disclosed herein can also be used to evaluate or characterize structure and function of a neuroactive pharmaceutical agent. For example, such methods allow assessment of activity (e.g., in terms of specificity, efficacy, etc. and/or to modulate the activity, by assaying or screening derivatives of said candidate compounds and comparing the activity of such derivatives to a parent unmodified modulator. For example, a chemical entity may be modified structurally by homologation with additional atoms, functional groups and/or substituents, or via substitution of atoms or groups, as will be appreciated by those skilled in the art.

Accordingly, the present disclosure provides methods and compositions for screening, identifying, characterizing, and modifying neuroactive compounds, for example, modulators or compounds that are active on or modulate neuronal cell function(s) and to identify and/or characterize and/or improve compounds which may be active on or modulate neurons. Such modulators or compounds may be useful for treating disorders of the nervous system wherein neural progenitor cell or neural cell function and/or behavior (e.g., proliferation and differentiation) may be implicated. Thus, in certain embodiments, the present methods may be employed to identify and/or characterize and/or improve compounds which able to modulate differentiation of neural progenitor cells (NPCs) into neurons.

Neuroactive pharmaceutical agents, compounds or modulators as described herein may also include any compound having the ability to alter (e.g., restore or correct) one or several functions of a cell (specifically a neuron or neural progenitor). For example, the compound of modulator may be capable of altering at least one metabolic pathway or biological or functional property of a cell (neuron) and to identify and/or characterize and/or improve compounds which are active on neurons and specifically able to modulate the differentiation of neural progenitor cells (NPCs) into neurons. As an example, a biologically active compound of this invention is a compound, which is capable of restoring a normal phenotype to an injured neuron or of at least partially inhibiting the deleterious effect of an injury on a neuron. In specific embodiments, the active compound may be selected for its capacity to repress or to activate a cellular mechanism, for its capacity to stimulate or inhibit a metabolic pathway, to restore a biological property, to prevent cell death, etc.

Pharmaceutical agents suitable for in vivo analyses may include, for example, morpholinos for knockdown of GOIs, enabling analysis of the gene's function in neurogenesis. Other pharmaceutical agents may include shRNA constructs. shRNA-mediated knockdown offers an independent method for knockdown compared to MOs and permits cell-type specific manipulation of protein expression. Methods have been developed to enhance shRNA-mediated knockdown in Xenopus neurons, and plasmid cassettes are available to streamline the generation of many shRNA constructs. Accordingly, shRNA constructs against GOIs can tested for specific knockdown of GOI expression (Chen et al., Front Neurosci 2009, 3: 63) and subsequently tested, for effects on NPC proliferation, for example. Of course, the methods or protocols as described herein may be employed for screening (or identifying, characterizing or improving) compounds that are active on any other attribute of neurogenesis, such as cell survival, NPC differentiation into neurons or glia, and the migration and assembly of cells within a brain region or neural circuit.

Methods and protocols as described herein may be employed for screening (or identifying, characterizing or improving) compounds that are active on neuronal survival or development, and which may specifically modulate the differentiation of NPCs into neurons.

### Pharmaceutical Compositions

In another aspect, the present invention provides pharmaceutical compositions comprising a pharmaceutical agent, modulator or compound as described identified by the methods of the present invention. Such pharmaceutical compositions may comprise pharmaceutical agents as described herein that can, for example, modulate neurogenesis or neuronal cell function(s), modulate NPC differentiation and/or proliferation, or may be active on or modulate neurons, and may be useful for treating disorders of the nervous system wherein neural progenitor cell or neural cell function and/or behavior (e.g., proliferation, differentiation, etc. . . . ) may be implicated.

Pharmaceutical agents as described herein can be formulated as pharmaceutical compositions and administered to, for example, a mammalian host such as a human patient in a variety of forms adapted to the chosen route of administration, e.g., orally or parenterally, by intravenous, intramuscular, topical or subcutaneous routes, and may comprise one or more pharmaceutically acceptable excipients.

### Cell-Based Assays

The methods of the present invention further comprise contacting isolated cells (e.g., neural progenitor cells) or suitable cultured cell lines with one or more candidate compounds or modulators. The cells may be contacted for various periods of time, depending on their effect, concentration, the cell population, and/or the evaluation technique. In a specific embodiment, the cells may be exposed to candidate compound(s), for example, in the range from about 1 nM to about 1 mM. It should be understood that other concentrations may be tested without deviating from the instant application. Furthermore, each compound may be tested, in parallel, at several concentrations. Furthermore, if necessary, different adjuvants and/or vectors and/or products helping the compounds to penetrate the cells may be added, including liposomes, cationic lipids or polymers, penetratin, Tat PDT, peptides from adenoviruses (e.g., penton or fiber) or other viruses, etc. Contacting can be performed in any appropriate support or device, such as incubation chambers for live Xenopus tadpole preparations.

Determining the profile of the candidate compounds can be performed according to several methods. In particular, different end points may be measured, in order to assess the neuroactivity of the compounds, such as: cell number, survival, expression of antigens, transcription of specific genes, and morphological changes, e.g., size, neurite growth, etc.

In a specific embodiment, the neuroactivity of the candidate compounds may be determined by comparison with control neuronal cell populations, in the absence of any compound and/or treated with reference compounds. Determining the status of the neurons can be performed by evaluating different physical measurements, for example, optical properties, fluorescence at various wavelengths, luminescence etc. Different instruments may be used, including automated microscopes fitted with lamps or lasers, etc. Other techniques include light detection through a refrigerated CCD camera. The signals measured may be treated according to known techniques, using for instance software including pixel histogram, cluster analysis and morphology analysis.

The present invention also relates to the use of any compound (or derivatives thereof) identified, selected, profiled or characterized by the methods of the present invention as, for example (i) targets for experimental research and/or (ii) manufacture of pharmaceutical compositions as modulators and specifically for treating neurological disorders.

In some embodiments, cell differentiation modes can be modulated by contacting the cells with a modulating agent. In other embodiments, cell differentiation modes can be modulated by introducing a modulating agent into the cells of interest by, e.g., electroporation, or any other suitable technique for introducing the modulating agent into the cells.

In specific embodiments, the pharmaceutical agent may increase the number or proportion of cells that divide in a proliferating mode relative to other division modes. In other embodiments, the pharmaceutical agent may decrease the number or proportion of cells which divide in a proliferating mode. In other embodiments, the pharmaceutical agent may increase the number or proportion of cells which divide in a mixed mode. Alternatively, the pharmaceutical agent may decrease the number or proportion of cells which divide in a mixed mode. In additional embodiments, the pharmaceutical agent may increase the number or proportion of cells which divide in a differentiating mode. Alternatively, the pharmaceutical agent may decrease the number or proportion of cells which divide in a differentiating mode. Combinations of pharmaceutical agents may also be employed to achieve a desired effect on NPC proliferation and differentiation.

The pharmaceutical agents of the present invention may be selected such that it modulates a known gene target implicated in the regulation of NPC proliferation and differentiation. Accordingly, a pharmaceutical agent (e.g., a morpholino, siRNA, etc.) may be constructed or selected to, for example, inhibit or upregulate translation of a target gene known to have a regulatory role in NPC proliferation and differentiation. Contacting the target cell(s) with or introducing such a pharmaceutical agent into the target cell(s) can thus effect modulation of NPC behavior.

In vivo Assays

The presently disclosed methods further comprise contacting isolated cells (e.g., neural progenitor cells) or suitable cultured cell lines with one or more candidate compounds or modulators. The cells may be contacted for various periods of time, depending on their effect, concentration, the cell population, and/or the evaluation technique. Generally, the cells are exposed to candidate compound(s) in the range from 1 nM to 1 mM. It should be understood that other concentrations may be tested without deviating from the instant application. Furthermore, each compound may be tested, in parallel, at several concentrations. Furthermore, if necessary, different adjuvants and/or vectors and/or products helping the compounds to penetrate the cells may be added, including liposomes, cationic lipids or polymers, penetratin, Tat PDT, peptides from adenoviruses (e.g., penton or fiber) or other viruses, etc.

Determining the profile of the candidate compounds can be performed according to several methods. In particular, different end points may be measured, in order to assess the neuro-activity of the compounds, such as: cell number, survival, expression of antigens, transcription of specific genes, and morphological changes, e.g., size, neurite growth, etc.

Preferably, the neuro-activity of the candidate compounds is determined by comparison with control neuronal cell populations, in the absence of any compound and/or treated with reference compounds. Determining the status of the neurons can be performed by evaluating different physical measurements, optical properties, fluorescence at various wavelengths, luminescence etc. Different instruments may be used, including automated microscopes fitted with lamps or lasers, etc. Other techniques include light detection through a refrigerated CCD camera. The signals measured may be treated according to known techniques, using for instance software including pixel histogram, cluster analysis and morphology analysis.

The invention also encompasses the use of any compound (or derivatives thereof) identified, selected, profiled or characterized by the above disclosed methods, (i) as targets for experimental research or (ii) for the manufacture of pharmaceutical compositions for treating neurological disorders.

In some embodiments, cell differentiation modes can be modulated by contacting the cells with a modulating agent. In other embodiments, cell differentiation modes can be modulated by introducing a modulating agent into the cells of interest by, e.g., electroporation, or any other suitable technique for introducing the modulating agent into the cells.

In certain embodiments, the pharmaceutical agent may increase the number or proportion of cells which divide in a proliferating mode relative to other division modes. In other embodiments, the pharmaceutical agent may decrease the number or proportion of cells which divide in a proliferating mode. In still other embodiments, the pharmaceutical agent may increase the number or proportion of cells which divide in a mixed mode. Alternatively, the pharmaceutical agent may decrease the number or proportion of cells which divide in a mixed mode. In additional embodiments, the pharmaceutical agent may increase the number or proportion of cells which divide in a differentiating mode. Alternatively, the pharmaceutical agent may decrease the number or proportion of cells which divide in a differentiating mode. Combinations of pharmaceutical agents may also be employed to achieve a desired effect on NPC proliferation and differentiation.

The pharmaceutical agent may be selected such that it modulates a known gene target implicated in the regulation of NPC proliferation and differentiation. Accordingly, a pharmaceutical agent (e.g., a morpholino, siRNA, etc.) may be constructed or selected to, e.g., inhibit or upregulate translation of a target gene known to have a regulatory role in NPC proliferation and differentiation. Contacting the target cell(s) with or introducing such a pharmaceutical agent into the target cell(s) can thus effect modulation of NPC behavior.

In vivo Assays

Analysis of genomic data generated by microarray and/or other genomic analysis techniques may be used to prioritize genes that can regulate proliferation and differentiation of NPCs in the tadpole CNS. Antisense morpholino oligonucleotides (MOs) (Eisen et al., Development 2008, 135(10): 1735-43) can thus be generated against genes of interest (GOI) and each GOI screened by morpholino-mediated knockdown for an effect of cell proliferation using imaging to assess BrdU incorporation in the CNS. For GOI that increase or decrease cell proliferation, several in vivo assays may be employed to test their function in controlling neurogenesis in the optic tectum (Nedivi et al, Science 1998, 281: 1863-1866; Ewald et al, J Neurosci 2008, 28(4): 850-61; Cantallopset al., Nat. Neurosci. 2000, 3: 498-503; Javaherian et al., Neuron, 2009 (in press); Van Aelst et al., Curr Opin Neurobiol 2004, 14(3): 297-304; Van Keuren-Jensen et al., Dev Neurobiol 2008, 68(11): 1315-24; Wu et al., Science 1998, 279: 222-226; Cline et al., J Physiol 2008, 586(6): 1509-17).

A first exemplary assay combines BrdU incorporation with immunolabeling for neuronal markers. The advantages of BrdU labeling are that the method is non-invasive and can be used as a relatively high-throughput screen of the effects of GOI knockdown or overexpression on cell proliferation. Combining BrdU and neuronal labeling allows quantitative evaluation of GOI on neurogenesis. Tadpoles can be exposed to a rearing solution containing BrdU. This method efficiently labels proliferative cells and allows greater control over BrdU exposure time. It has been shown that access of BrdU does not change over the developmental periods studied and that BrdU incorporation does not occur in response to DNA damage. BrdU exposures are typically performed at the same time of day for all animals to control for potential circadian effects on proliferation. After BrdU exposure, animals are either fixed immediately, or reared for 2-3 days before fixing and processing animals for BrdU immunolabeling.

Animals are then terminally anesthetized, microwave fixed (Paupard et al., J. Histochem. Cytochem. 2001, 49(8): 949-956) and brains are processed for antibody labeling in wholemount or vibratome sections (Peunova et al., 8809-8818) with primary antibodies (mouse anti-BrdU, BD Bioscience; rabbit anti NeuroD; Abcam and Alexafluor secondary antibodies (Molecular Probes)).

A second exemplary assay uses in vivo time-lapse imaging of cells labeled with suitable reporters or markers, such as Sox2.mFGF4::FP-labeled NPCs. The advantages of time-lapse in vivo imaging experiments are that longitudinal studies allow direct observation of cell proliferation, cell fate and structural dynamics underlying these events. How GOI overexpression/knockdown alters neurogenesis in vivo is quantified, and cell division rates and fate of progeny can be determined.

Proliferative cells in ventricular sections, whole mount fixed brain preparations and in vivo FP-labeled samples can be imaged using a spinning disk confocal attachment mounted on a microscope equipped with laser lines and dichroic mirrors and filters to resolve UV to far red fluorophores. Signals are captured on a sensitive, high signal to noise EMCCD camera. Images are acquired using appropriate image acquisition software. In double-label experiments, images are acquired sequentially to eliminate bleed-through. Controls with single fluorophore labelings are done to ensure absence of bleed-through.

For in vivo image time-lapse imaging, anesthetized tadpoles are placed in a custom imaging chamber. Complete confocal stacks at 1 μm z-step are acquired using proper laser/filter settings for the fluorophores. For fixed tissue whole mounts and sections, complete z-stacks (0.5 μm z-interval) are acquired of the tectal lobes.

For analysis of time-lapse data, radial glia and neurons are distinguished according to morphological criteria based on the 3 dimensional structure of the cells. The numbers and sequence of symmetric, proliferative divisions; asymmetric, neurogenic divisions; terminal symmetric neurogenic divisions, and the differentiation state of each cell in the lineage over the timecourse of the experiment are determined. Time-lapse imaging of the individual NPCs allows identification of the fate of cells over the course of the imaging experiment, including the assessment of morphological changes in dividing and differentiating cells.

Thus, in certain embodiments the present methods are useful for screening (or identifying, characterizing or improving) pharmaceutical agents that alter the differentiation of neural progenitor cells (NPCs) into neurons in vivo, more particularly in an intact brain. An advantage of the present methods is that such methods may employ neural cell populations in vivo, such as those in the intact visual system of a Xenopus tadpole. Accordingly, the use of in vivo neural cell populations allows a predictive and reliable assessment of the biological activity of a compound or modulator. The NPCs and neurons employed may be of various origins, including mammalian origin (e.g., rodents, humans, primates, etc. . . . ) as well as amphibians such as Xenopus laevis.

The activity of a pharmaceutical agent in vivo can be determined, for example, as described herein with respect to screening of genes related to neurogenesis. For example, since Xenopus tadpoles are amenable to in vivo time-lapse imaging, neural progenitor cells and their progeny can be imaged in the intact animal. Accordingly, after contacting NPC cells in vivo with a candidate pharmaceutical agent, the rate of proliferation in a test animal population relative to a control animal population may be measured (e.g., visualized), and the activity of a candidate modulating determined by the relative rates of NPC proliferation in animals treated or contacted with a candidate pharmaceutical agent relative to a control population. A pharmaceutical agent that modifies NPC proliferation rates (e.g., enhances or decreases) is thus identified as a modulator of neurogenesis and neural cell proliferation.

Conditions described herein with respect to in vitro methods for screening of pharmaceutical agents as modulators of neurogenesis and gene screening methods in vivo (e.g., concentration of the pharmaceutical agent, readout, etc.) may also be employed here. For example, an NPC cell population in an intact animal CNS region (e.g., optic tectum) may be transfected (e.g., by electroporation) so its expresses a fluorescent protein (FP). FP-expressing cells in the intact animal can be imaged at time t=0, then imaged again after a predetermined time period (e.g., 24 h, 48 h, 96 hr, etc.), and may be imaged at intervals during the predetermined time period (e.g., at 12 hr, 24 hr, 36 hr, etc.). The change in relative type and number of cells (e.g., NPCs and differentiated neurons) over the predetermined time period each 24 h interval can be determined by classifying the cells according to their morphologies. Changes in morphology may be characterized as fractions of NPCs and glial cells in a test population relative to a control population. Differences between the cells in the test animal population and the control animal population may indicate that the candidate agent is a modulator of NPC proliferation.

In a specific embodiment, a screen of GOI effects on CNS cell proliferation can be perfomed using an imaging assay to assess BrdU incorporation in X. laevis tadpoles whose brains were electroporated with morpholinos to GOIs. One to two days later, proliferative cells may be labeled by exposure to BrdU for 2 hr before sacrifice. Brains can then be processed to detect BrdU in wholemount and imaged by collecting a complete z series of confocal images through the brain. Such imaging of wholemount brains provides an excellent method to quantify levels of cell proliferation.

### EXAMPLES

The present disclosure will be further illustrated by the following non-limiting Examples. The following Examples are understood to be exemplary only, and should not be construed as limiting the scope of the invention as defined by the appended claims.

### 1. Visual Stimulus and NPC Proliferation

### Overview

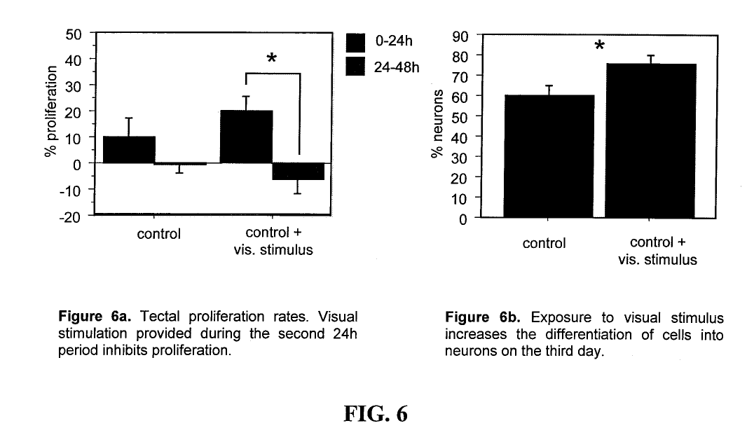

Neural progenitor cell (NPC) proliferation and differentiation were assayed in the visual system of the intact Xenopus tadpole central nervous system. This experimental system allows manipulation of neural activity by exposing animals to visual system stimulation or depriving animals of visual system stimulation. The results show that the rate of proliferation of NPCs is increased in animals deprived of visual stimulation compared to animals reared under conditions of 12 h light/12 h dark. Animals which are deprived of visual stimulation for 24 h followed by a 24 h period of visual stimulation show an increased rate of proliferation during the first 24 h in the absence of visual experience, followed by differentiation of the majority of new generated cells (FIG. 6). These data show that manipulating neural activity control both proliferation rate and differentiation: decreased neural activity increases proliferation and increased neural activity triggers the differentiation of progenitors into neurons.

### Methods

NPCs were transfected in intact Xenopus tadpoles so they express fluorescent protein (FP). FP-expressing cells in intact animals were imaged using confocal microscopy. After imaging the animals were then placed in light-tight chamber so they received no visual stimulation over the next 24 h. FP-expressing cells were imaged again and the animals were placed in a chamber where they received visual stimulation for 24 h. Animals were imaged for a third time. The change in cell numbers over each 24 h interval and the identity of the cells as radial glia (NPCs) or neurons was determined according to their morphologies. Data are expressed as the change in cell number per 24 h and as the fraction of cells with NPC or neuronal morphologies.

### Results

In the absence of visual experience, cell numbers increase +19.9±5.8% over a 24 hour period (N=12 tecta analyzed). After exposing the animals to the visual stimulus for 24 hours, we find a significant 6.3±5.4% drop in cell numbers (Wilcoxon singed-rank test, p=0.01). This negative rate indicates that the cells have left the proliferative cycle because the fluorescence marker used to detect the cells is driven by a promoter that is expressed only during proliferation. A separate set of animals that were not exposed to the visual experience showed no significant differences in the proliferation rate between the two 24 hour periods (N=7 tecta analyzed, p=0.4).

There was a steady rate neurogenesis in the brains of animals which were not exposed to the visual stimulus. In the absence of visual stimulation, the rate of new neurons increased 32.9±11.3% over the first 24 hour interval which was not significant from the 30.1±9.4% increase measured over the second 24 hour interval (N=7 tecta, Wilcoxon signed rank test p=0.73). In contrast, the neurogenesis rate of animals that were exposed to the visual stimulus during the last 24 hour interval fell significantly from 79.4±22.9% in the first 24 hour interval to 18.0±9.4% during the second 24 hour interval (N=12 tecta, p=0.05).

In the absence of the 24 hour visual experience, the rate that cells lose their glial appearance shows a 17.1±9.7% drop during the first 24 hour period and a 44.9±10.6% decrease during the last 24 hour interval (N=7 tecta, p=0.18). The rate that cells from the animals with 24 hours of visual deprivation followed by 24 hour exposure to visual stimulation lose their glial appearance significantly increases after exposure to the visual stimulus (17.1±9.7% loss before and 56.0±10.0% loss afterward, p=0.04).

Together these results are consistent with visual experience disrupting cell proliferation and increasing the rate with which glial precursors differentiate into neurons.

2. Modulating Agents with and without Visual Experience and NPC Proliferation

### Background and Methods

### NPC Detection

These experiments took advantage of a cell-type specific, fluorescent reporter system that drives protein expression in dividing cells (see FIG. 3). The reporter consisted of 6 repeats of upstream regulatory elements of fibroblast growth factor 4 (FGF4). Endogenous sox2/oct3 transcription factors bind to the FGF4 regulatory elements and drive the expression of Gal4 which in turn drives the UAS-fluorescent protein. FGF4, sox2 and oct3, are each expressed in proliferating cells, and relying on endogenous sox2/oct3 transcription factors to drive Gal4 promoted specificity of reporter expression within proliferating cells. The UAS-fluorescent protein was expressed as a separate construct; this reporter added modularity and specificity to this reporter system.

Tectal cells expressing the above-described fluorescent reporter were harvested from tadpoles with varying visual experience as well as from tadpoles that had expressed the construct for different amounts of time. RNA from these cells was then processed and microarray comparisons made. Identified genes of interest selected for further analysis are summarized in Table 1 below:

TABLE 1
Genes of Interest Identified via Microarray Analysis

| Accession No. | Actual name | Description |
|---|---|---|
| NM 001092639.1 | MGC82106 | A putative isoform of Elk4. |
| | ELK4, ETS-domain protein (SRF | |
| | accessory protein 1) (elk4-b) | |
| BC170068.1 | Polysomal ribonuclease 1 | |
| BC170070.1 | Polysomal ribonuclease 1 | |
| BC169356.1 | cAMP-dependent protein | |
| | kinase catalytic subunit alpha | |
| | Cytoplasmic polyadenylation | RNA-binding protein that stimulates |
| | element binding protein 1 | polyadenylation and translation in |
| | | germ cells and neurons |
| | Candidate plasticity gene 15 | Involved in cell survival and is |
| | | regulated by activity. |
| BC106400.1 | Deiodinase, iodothyronine, type | Removes iodine from the active form |
| | III | of thyroid hormone (T3) to inactivate |
| | | it. |
| NM 001087027.1 | Xenopus laevis ephrin-A3 | |
| | (efna3) (ligand) | |
| NM 001085854.1 | ELK4, ETS-domain protein (SRF | A transcription factor and member of |
| | accessory protein 1) (elk4-a) | the ternary complex factors. Regulated |
| | (sap1) | by extracellular regulated kinases as |
| | | part of the MAPK pathway of growth |
| | | factor signaling. |
| NM 001090601.1 | Ephrin-B1 | A type I membrane protein and a |
| | EPH receptor B1 | ligand of Eph-related receptor tyrosine |
| | | kinases. |
| NM 001085687.1 | Fragile X mental retardation | RNA binding protein whose loss results |
| | protein 1 | in Fragile X mental retardation |
| NM 001088317.1 | Fragile X mental retardation, | Known to interact with fmr1A |
| | autosomal homologue gene | |
| NM 001088783.1 | Glutathione S-transferase pi 1 | Part of the family of proteins that |
| | | catalyze the conjugation of |
| | | hydrophobic and electrophilic |
| | | compounds with reduced glutathione. |
| NM 001087017.1 | Histone deacetylase 6 | Responsible for the deacetylation of |
| | | histones and epigenetic changes in |
| | | chromosomes. |
| NM 001086595.1 | Heat shock protein | Member of the heat-shock protein-70 |
| | (glucose-regulated protein 78 | (HSP70) family regulated by glucose |
| | (GRP78) | levels. |
| NM 001086503.1 | Matrix metalloproteinase 9 | A TypeIV collagenase involved in the |
| | | breakdown of extracellular matrix. |
| BC169513.1 | Wingless-type MMTV | The Wnt genes are ligands that bind |
| | integration site family, member | members of the frizzled family of |
| | 7B | seven transmembrane receptors. |

Comparisons were made between tadpoles of different ages (greater population of proliferating cells versus greater population of terminally differentiated cells) and tadpoles with different visual experience, which may also bias the relative levels of proliferations versus terminal differentiation. Genes that showed significant changes in their expression in multiple microarray comparisons were prioritized.

The above-described reporter was employed to drive the fluorescent protein Kaede in the proliferating cells of the optic tectum of tadpoles. The fluorescent emission spectrum of Kaede can be photoconverted from green to red after exposure to a UV light or 405 nm laser source, which added temporal control to the experiments. 24-36 hours after the tadpoles were transfected with the reporter, all the Kaede-expressing cells were photoconverted to the red form of the protein. Based on published data using similar methods (Caron et al., Development 2008, 135:3259-3269), newly produced progeny inherit the red Kaede during cytokinesis, but continue to synthesize new green Kaede protein. For example, Caron et al. (ibid.) identified newly generated cells by the relative levels of green to red.

Cells of the optic tectum were transfected with the proliferation reporter by electroporation, a well-established method that reliably results in multiple labeled cells in the tissue (Haas et al., Differentiation 2002, 70: 148-154). The Gal4 driver and UAS-kaede plasmids (0.5 μg/μl) were injected into the ventricle of the optic tectum and then voltage pulses were applied across the tissue to drive the plasmids into the cells of the tectum. These plasmid constructs were co-electroporated with antisense morpholino oligonucleotides designed to inhibit the translation of the candidate genes (Eisen et al., Development 2008, 135: 1735-1743). The morpholinos were electroporated at 0.1 mM and visualized by a lissamine fluorescent tag. To prevent conversion of Kaede from UV wavelength from ambient light, the animals were kept in the dark. 24-36 hours later, the tadpoles were anesthetized and a complete z-stack through the optic tectum of each animal was collected on each day for 3 consecutive days. After the first imaging of the tectum, all animals were returned to the dark for the 24 hour period until the second imaging. At that time, some animals were exposed to an enhanced visual environment which consisted of a chamber with an array of LED lights (emitting at 567 nm, beyond the wavelength for converting the Kaede protein) which flashed for 1 sec. at 0.2 Hz. This visual stimulus has been shown to enhance the synaptic drive onto the tectal neurons and results in significant changes in synaptic plasticity (Sin et al., Nature 2002, 419: 475-480).

Data Acquisition and Analysis

Acquisition settings used on the first day were chosen to protect against pixel values reaching saturation by the third day. The selected settings were used throughout the experiment. Volocity software (Improvision, Perkin Elmer) which uses the 3D information from the acquired z-stacks was used to identify and select cell bodies of the labeled cells based on the standard deviation of the intensity and size of the objects. The identified objects were then experimenter verified and the cell type (glia, neuron, or undefined) was assigned based on cell morphology. From each tectal lobe typically between 15 and 45 cells were transfected. Percent proliferation was calculated as the change in cell numbers per 24 hour period. These measurements are reported as mean±s.e.m.

The analysis of proliferation in control animals, animals with cell proliferation blockers, and animals which received the 24 hour enhanced visual stimulation are reported. Results of experiments in which 2 candidates identified from the microarrays (Dio3 and GstPi) were knocked down with antisense morpholinos are also reported. In addition, results from experiments to determine visual experience affects proliferation in the presence of Dio3 morpholino knockdown are reported. The experimental conditions employed are summarized in Table 2 below:

TABLE 2
Morpholino Knockdown Experiment Conditions

| | | Visual | N |
|---|---|---|---|
| Morpholino | Description | stimulus | (tectal lobes) |
| — | Proliferation Reporter + DMSO | No | 8 |
| — | Proliferation Reporter + aphidicolin and hydroxyurea | No | 10 |
| — | Proliferation Reporter only | No | 12 |
| — | Proliferation Reporter only | Yes | 12 |
| Dio3 | Deiodinase, iodothyrine, type III | No | 5 |
| Dio3 | Deiodinase, iodothyrine, type III | Yes | 4 |
| Gst Pi | Glutathione S-transferase Pi | No | 6 |
| HDAC6 | Histone deacetylase 6 | No | — |
| Wnt7b | Wingless-type MMTV (murine mammary tumor virus) | No | — |
| | integration site family, 7b | | |
| HSPA5 | Heat shock protein 70 | No | — |
| MMP9 | Matrix metalloproteinase, a type IV collagenase | No | — |
| FXR1 | Fragile X mental retardation, autosomal homologue | No | — |
| Fmr1A | Fragile X mental retardation protein 1 | No | — |

### Results and Discussion

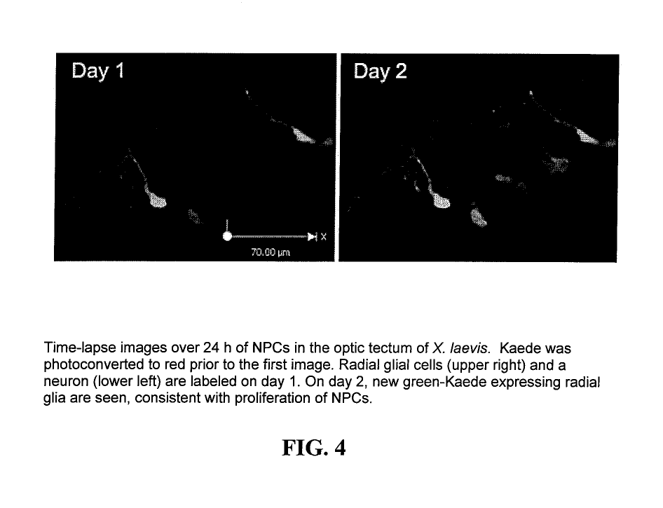

Expression of the Sox2 reporter construct demonstrated that radial glia are the major neural progenitor cell in the Xenopus optic tectum (FIG. 4). Neurons did not continue to express green Kaede after differentiation since differentiated cells no longer produce sox2/oct3 to drive the gal4-UAS-Kaede consturct. The majority of NPC divisions were found to be terminal divisions.

Cell Division Blockers

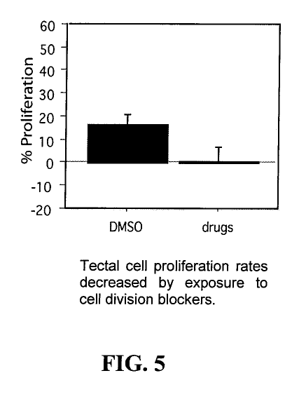

Aphidicolin (150 uM) and hydroxyurea (20 mM in 2% DMSO) were applied to cells to block cell divisions, and the proliferation rates of those cells compared with control animals receiving only DMSO. This treatment disrupts proliferation in Xenopus (Harris et al., Neuron 1991, 6: 499-515). In control animals exposed to DMSO only, an average increase of 16.4±4.2% in cell number over 24 hours (N=8 tectal lobes) was observed. Aphidicolin and hydroxyurea blocked cell proliferation (increase in cell numbers: 0.41±6.28% (N=10, p=0.1, Mann-Whitney; see FIG. 5).

Visual Experience-Dependent Control of NPC Fate

In the absence of visual experience, cell numbers increased 19.9±5.8% over a 24 hour period (N=12 tectal lobes). After exposing the animals to the visual stimulus for 24 hours a significant 6.3±5.4% decrease in cell numbers was found (p=0.01). This negative rate shows that the cells may have left the proliferative cycle and are no longer detectable due to dilution of the red Kaede reporter in the increasingly growing dendritic arbor. The Kaede reporter is expressed only during proliferation and accordingly, terminally differentiated cells will exhibit only that reporter produced while in proliferative mode. Control animals which did not receive the 24 hour period of visual stimulation showed no significant differences in the proliferation rate between the two 24 hour periods (10.3±7.2% and −0.6±3.5%; N=12 tectal lobes analyzed, p=0.23; FIG. 6A).

To test whether exposure to visual experience affects the fate of cells in the tectum, the fraction of neurons in the labeled population after 24 h with or without visual stimulation was compared. The proportion of cells with neuronal morphology was greater in animals that were exposed to visual stimulus as compared to control animals (75.7±4.1% versus 60.1±4.8%, p=0.02, Mann-Whitney; FIG. 6B), showing that visual experience promotes differentiation of NPC progeny.

Dio3-Morpholino Expression and Neurogenesis

Deiodinase iodothyronine type III is the enzyme in the thyroid hormone pathway which removes iodine from the active form of thyroid hormone (T3), effectively inactivating it. A microarray analysis suggested that Dio3 expression was increased in active progenitor cells. T3 levels in X. laevis are low before metamorphic stages, but the presence of T3 receptors has been detected in NPCs, suggesting that relative changes in T3 levels may affect proliferation. Increased proliferation correlates with increased thyroid hormone and receptor activation in X. laevis tadpoles at metamorphic stages (Denver et al., Dev Biol 2009, 326: 155-168). Therefore, a knockdown of Dio3 with morpholino expression should increase T3 levels and accordingly increase proliferation.

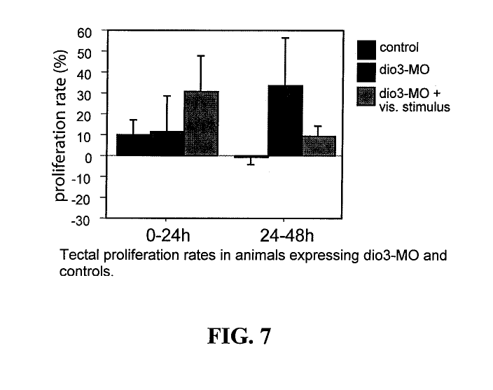

Animals transfected with morpholinos against Dio3 (Dio3-MO) exhibited increased cell numbers: 11.4±17.3% in the first 24 hour period and 33.9±22.2% over the second 24 h period (N=4, FIG. 7). In contrast, control animals show the largest increase in proliferation the first 24 hour period, then little proliferation over the subsequent 24 hour period. Unusual clusters of cells that appear to be clones were found within the tecta of animals transfected with Dio3-MO, consistent with unusual patterns of proliferation without the normal time for migration.

Some animals expressing Dio3-MO were also exposed to visual stimulus during the second 24 h period (FIG. 7). Visual experience was found to decrease rates of proliferation in animals transfected with Dio3-MO (N=5), however this difference was not considered significant due to the large variation in proliferation rates.

Knockdown of GSTpi Promotes Neuronal Differentiation

Glutathione S-transferase Pi 1 (GSTP1) is a member of the Glutathione S-transferase family of proteins, which plays an important role in detoxification by catalyzing the conjugation of many hydrophobic and electrophilic compounds with reduced glutathione. GST-Pi 1 is thought to play a role in the susceptibility to cancers. GST-Pi 1 was observed to be up-regulated in neural precursor cells and therefore a GST-Pi knockdown is predicted to decrease proliferation rates.

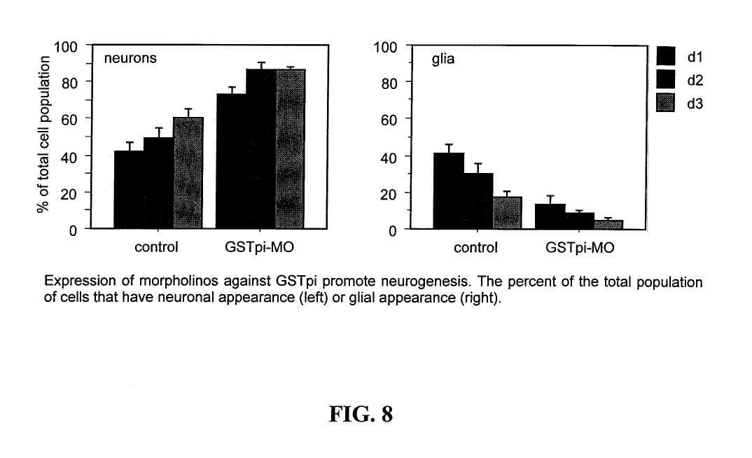

A striking result was observed in the tecta of tadpoles expressing morpholinos against GSTpi. The number of neurons even by the first day was significantly greater than that observed in control animals (FIG. 8). A significantly larger population of tectal cells (72.7±4.7%, N=7 tectal lobes) expressing GSTpi-MO were differentiated into neurons by day 1 in test animals relative to control animals (42.0±4.7%), (p=0.002, Mann-Whitney). By day 2, 86.4±3.7% of the cells have differentiated into neurons in the test animals compared to 49.2±5.7% within the control population (p=0.001). This proportion of neurons may represent a maximum as the number of neurons was not observed to increase in the test animals over the third period (86.8±1.0%), and is significantly larger than the proportion of neurons within the control animals (60.1±16.6%; p=0.004).

An increase in the total proportion of neurons shows that the neural precursor population decreases with GSTpi-MO expression. The proportion of radial glia decreases over the 3 day period in animals expressing GSTpi-MO, as well as control animals (FIG. 8). Comparing the proportion of cells from brains expressing GSTpi-MO to the control animals showed that by day 1 there were significantly fewer glia (13.3±4.6% and 41.5±4.8%; p=0.005) and these differences continued over the next two days (8.4±2.0% and 17.4±3.5%, p=0.02; 4.8±1.6% and 13.3±4.6%, p=0.02). A small proportion of cells could not be categorized into radial glia or neurons.

Fragile X Related Genes

Fmr1A. Fragile X mental retardation protein 1 is an mRNA binding protein that is thought to regulate mRNA trafficking from the nucleus to the cytoplasm and local protein translation within neurons. Microarray data suggested that expression of FMR1 and a protein similar to 82-kD FMRP Interacting Protein, proliferation-inducing gene 1 (AKA nuclear fragile X mental retardation protein interaction protein) were lower in NPCs compared to differentiated neurons. The potential role of FMRP and related proteins in neuronal proliferation is not entirely clear. One study showed that FMR1 increases NPC proliferation and alters differentiation (Castren et al., Proc Natl Acad Sci USA 2005, 102: 17834-17839), while another showed that FMR1 only alters NPC differentiation (Bhattacharyya et al., Stem Cells Dev 2008 17: 107-117). The possible discrepancy between these studies may be due to the fact that each of the studies uses a different source of in vitro cells. An in vivo study may clarify the role of FMR1 and related genes in proliferation of NPCs.

FXR1. Fragile X mental retardation, autosomal homologue gene interacts with the functionally-similar proteins FMR1 and FXR2. Based on microarray data, knocking down FXR1 may increase proliferation by inhibiting differentiation.

Morpholinos against FMR1A and FXR1 appeared to decrease NPC proliferation in the tectum. These were qualitative observations.

What is claimed is:

1. A method, comprising:
a) contacting neural progenitor cells in an intact brain region of a first animal with a pharmaceutical agent;
b) exposing said first animal and a second control animal to a visual stimulus; and
c) measuring proliferation rates of said neural progenitor cells in said first animal and of neural progenitor cells in said second animal;
wherein a difference in proliferation rate between said neural progenitor subject cells and said neural progenitor control cells identifies the pharmaceutical agent as one capable of modulating neural proliferation

2. The method of claim 1, wherein said intact brain region comprises the optic tectum.

3. The method of claim 1, wherein said intact brain region is involved in processing one or more of the group selected from olfactory inputs, visual inputs, and mechanosensory inputs, or is involved in mediating behavioral outputs.

4. The method of claim 1, wherein said intact brain region comprises circuits of the telencephalon, midbrain, hindbrain/spinal cord, retina, or olfactory pit.

5. The method of claim 1, wherein said first and second animals are Xenopus laevis.

6. The method of claim 1, wherein measuring comprises counting the number and type of cells in the optic tectum of said first and second animals.

7. The method of claim 1, wherein contacting said neural progenitor cells with pharmaceutical agent comprises electroporating said pharmaceutical agent into said neural progenitor cells.

8. A method, comprising:
a) contacting neural progenitor subject cells with a pharmaceutical agent in an amount effective to modulate expression of one or more genes in said neural progenitor subject cells;
b) measuring proliferation rates of said neural progenitor subject cells and of neural progenitor control cells that have not been contacted with said pharmaceutical agent; and
c) comparing the proliferation rates of said neural progenitor subject cells and said neural progenitor control cells;
wherein a difference in proliferation rate between said neural progenitor subject cells and said neural progenitor control cells identifies the one or more genes as modulators of proliferation of neural progenitor cells.

9. The method of claim 8, wherein said neural progenitor subject cells are in a first animal and said neural progenitor control cells are in a second animal.

10. The method of claim 9, wherein said neural progenitor subject and control cells are in an intact brain region of each of said first and second animals respectively.

11. The method of claim 10, wherein said intact brain region is involved in processing olfactory inputs, visual inputs, or mechanosensory inputs, or is involved in mediating behavioral outputs.

12. The method of claim 10, wherein said intact brain region comprises circuits of the telencephalon, midbrain, hindbrain/spinal cord, retina, or olfactory pit.

13. The method of claim 10, wherein said first and second animals are Xenopus laevis.

14. The method of claim 8, further comprising introducing a reporter construct into said neural progenitor subject cells and said neural progenitor control cells.

15. The method of claim 14, wherein said reporter construct comprises a gene encoding a fluorescent protein.

16. The method of claim 15, wherein the fluorescent protein is specifically expressed in neural progenitor cells.

17. The method of claim 14, wherein introducing comprises transfecting with a plasmid encoding said reporter construct.

18. The method of claim 8, wherein measuring comprises counting the number and type of cells before and after at least one predetermined time period.

19. The method of claim 8, wherein contacting said neural progenitor subject cells with a pharmaceutical agent comprises electroporating said pharmaceutical agent into said neural progenitor subject cells.

20. The method of claim 9, further comprising exposing said first and second animals to a visual stimulus.

21. The method of claim 8, wherein said pharmaceutical agent comprises a chemical compound or an antisense oligonucleotide.

22. The method of claim 21, wherein said antisense oliogonucleotide comprises an siRNA, and shRNA and/or a morpholino.

23. The method of claim 8, wherein said one or more genes are selected from SEQ. ID. NOs. 1-651, or functional truncations, modifications and/or substitutions thereof.

24. A method, comprising:
a) contacting neural progenitor subject cells with a pharmaceutical agent;
b) measuring proliferation rates of said neural progenitor subject cells and of neural progenitor control cells that have not been contacted with said pharmaceutical agent; and
c) comparing the proliferation rates of said neural progenitor subject cells and said neural progenitor control cells;
wherein a difference in proliferation rate between said neural progenitor subject cells and said neural progenitor control cells identifies the pharmaceutical agent as one capable of modulating proliferation.

25. The method of claim 24, wherein said neural progenitor subject cells are in a first animal and said neural progenitor control cells are in a second animal.

26. The method of claim 25, wherein said neural progenitor subject and neural progenitor control cells are in an intact brain region of each of said first and second animals.

27. The method of claim 26, wherein said intact brain region is involved in processing olfactory inputs, visual inputs, or mechanosensory inputs, or is involved in mediating behavioral outputs.

28. The method of claim 26, wherein said intact brain region comprises circuits of the telencephalon, midbrain, hindbrain/spinal cord, retina, or olfactory pit.

29. The method of claim 25, wherein said first and second animals are Xenopus laevis.

30. The method of claim 24, further comprising introducing a reporter construct into said neural progenitor subject cells and said neural progenitor control cells.

31. The method of claim 30, wherein said reporter construct comprises a gene encoding a fluorescent protein.

32. The method of claim 31, wherein the fluorescent protein is specifically expressed in neural progenitor cells.

33. The method of claim 30, wherein introducing comprises transfecting with a plasmid encoding said reporter construct.

34. The method of claim 24, wherein measuring comprises counting the number and type of cells before and after at least one predetermined time period.

35. The method of claim 24, wherein contacting said neural progenitor subject cells with a pharmaceutical agent comprises electroporating said pharmaceutical agent into said neural progenitor subject cells.

36. The method of claim 24, further comprising exposing said first and second animals to a visual stimulus.

37. A method, comprising;
a) administering a pharmaceutical agent to subject cells expressing a target gene selected from the group consisting of SEQ ID NOs. 1-651, or functional truncations, modifications and/or substitutions thereof; and
b) comparing expression of the target gene in the subject cells administered the pharmaceutical agent compared with expression of the target gene in subject cells not contacted with the pharmaceutical agent;
wherein a difference in expression of the target gene in subject cells administered the pharmaceutical agent compared with subject cells not administered the pharmaceutical agent identifies the pharmaceutical agent as a candidate modulator of neural proliferation or differentiation.

38. The method of claim 37, wherein the subject and control cells are neural progenitor cells.

39. The method of claim 37, further comprising evaluating the candidate modulator of neurogenesis in an intact brain region.

40. The method of claim 39, wherein the intact brain region is the optic tectum of Xenopus laevis.

41. A pharmaceutical agent identified by the method of claim 1.

42. A pharmaceutical agent identified by the method of claim 8.

43. A pharmaceutical agent identified by the method of claim 24.

44. A pharmaceutical agent identified by the method of claim 37.

45. A method, comprising administering the pharmaceutical agent of claim 38 to a patient.

46. A method, comprising administering the pharmaceutical agent of claim 39 to a patient.

47. A method, comprising administering the pharmaceutical agent of claim 40 to a patient.

48. A method, comprising administering the pharmaceutical agent of claim 41 to a patient.

49. A pharmaceutical composition comprising the pharmaceutical agent of claim 38.

50. A pharmaceutical composition comprising the pharmaceutical agent of claim 39.

51. A pharmaceutical composition comprising the pharmaceutical agent of claim 40.

52. A pharmaceutical composition comprising the pharmaceutical agent of claim 41.
